(19) United States
(12) Patent Application Publication
(10) Pub. No.: US 2018/0325793 A1
(43) Pub. Date: Nov. 15, 2018

(54) HIGH-PERFORMANCE COSMETIC PRODUCTS CONTAINING A POLYORGANOSILOXANE AND A DIPEPTIDE

(71) Applicant: Evonik Degussa GmbH, Essen (DE)

(72) Inventors: Erik Schulze zur Weische, Hamburg (DE); Volker Scheunemann, Lüneburg (DE)

(73) Assignee: Evonik Degussa GmbH, Essen (DE)

(21) Appl. No.: 15/525,793

(22) PCT Filed: Dec. 7, 2015
(86) PCT No.: PCT/EP2015/078776

§ 371 Date: May 10, 2017

(30) Foreign Application Priority Data
Dec. 9, 2014 (DE) 102014225209.1

(51) Int. Cl. A61K 8/64; A61K 8/58; A61K 8/34; A61Q 5/00; A61Q 5/02; A61Q 5/12

(52) U.S. Cl. CPC A61K 8/64; A61K 8/585; A61Q 5/12; A61Q 5/002; A61Q 5/02; A61K 8/342

(57) ABSTRACT

The present invention relates to cosmetic compositions, particularly hair treatment compositions, comprising a polyorganosiloxane of formula (I)

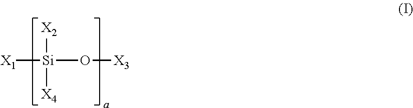

and methionylmethionine.

The cosmetic compositions with methionylmethionine and the polyorganosiloxanes selected show a positive effect on skin and hair and the hair follicle.

The invention relates to cosmetic compositions, particularly hair treatment compositions such as shampoos and so-called conditioners, having an active ingredient combination for mild and effective hair care.

Not least by intense stress on the hair, for example by dyes or perming and also by cleansing of the hair with shampoos and by environmental pollution, the importance of care products with the maximum long-lasting effect is increasing. Such care compositions influence the natural structure and properties of hair. For instance, following such care treatments, for example the wet and dry combability of the hair, the hold and the fullness of the hair can be optimized or the hair can be protected from increased split-ends.

Therefore, it has been customary for some time to subject the hair to a special after-treatment. Here, typically in the form of a conditioner, the hair is treated with specific active ingredients, for example, quaternary ammonium salts or special polymers. By means of this treatment, depending on the formulation, the combability, the hold and fullness of the hair are improved and the split-end rate decreased.

Multifunctional cosmetic products are also known from the prior art. These particularly include the so-called “2-in-1” shampoos, which not only cleanse the hair, but also condition it. Such products are highly valued by the consumer, since, due to their product performance, they eliminate at least one process step, conditioning with a classical hair conditioner for example.

Silicones and particularly amino-functional silicones are known as care substances in hair-treatment compositions and corresponding products are widespread in the market. Cosmetic compositions with amino-functional polyorganosiloxanes are known from WO 2013/079299 A2 which improve numerous properties of the body surfaces treated therewith, particularly hair, and lead to improved handling in addition to improved combability for example. There exists however further demand to improve the achieved effects, particularly with regard to the handling, the combability, the softness and the volume of the hair and hairstyling and to further develop cosmetic compositions in this regard.

It has now been found that a combination of certain polyorganosiloxanes with methionylmethionine has a particularly positive effect on body surfaces, particularly on the skin, the hair and the hair follicle.

A first subject matter of the present invention are cosmetic compositions comprising—in each case based on their weight—

a) 0.0001% to 20% by weight polyorganosiloxane of the formula (I)

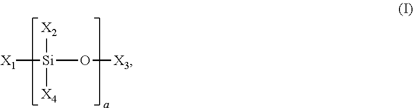

in which
X₁ and X₂ are mutually independently OH, OR¹, R², O-PDMS or O-fSiloxane,
X₃ is hydrogen or a monovalent hydrocarbon radical having 1 to 8 carbon atoms per radical, PDMS or fSiloxane,
X₄ is a radical of the formula

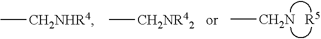

and
a is a number from 1 to 100,
where
R¹ is an alkyl radical having 1 to 8 carbon atoms,
R² is a monovalent saturated or unsaturated hydrocarbon radical having 1 to 200 carbon atoms per radical, optionally substituted with the elements N, P, S, O, Si and halogen, PDMS is

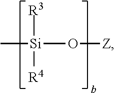

fSiloxane is

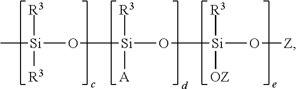

R³ is each mutually independently a monovalent saturated or unsaturated hydrocarbon radical having 1 to 200 carbon atoms per radical, optionally substituted with the elements N, P, S, O, Si and halogen,
A is a radical of the formula R⁶—[NR⁷—R⁸—]_{f}NR⁷₂,
where
R⁶ is a divalent linear or branched hydrocarbon radical having 3 to 18 carbon atoms,
R⁷ is a hydrogen atom, an alkyl radical having 1 to 8 carbon atoms or an acyl radical,
R⁸ is a divalent hydrocarbon radical having 1 to 6 carbon atoms,
b is a number from 1 to 2000,
c is 0 or a number from 1 to 2000,
d is a number from 1 to 1000,
e is 0 or a number from 1 to 5,
f is 0, 1, 2, 3 or 4,
Z is hydrogen, an alkyl radical having 1 to 8 carbon atoms or

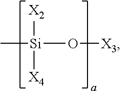

R⁴ is a monovalent hydrocarbon radical having 1 to 18 carbon atoms comprising optionally N and/or O atoms and
R⁵ is a divalent hydrocarbon radical having 3 to 12 carbon atoms comprising optionally N and/or O atoms, and
   b) 0.0001 to 30% by weight methionylmethionine.

The composition is a cosmetic composition. Preferred cosmetic compositions are selected from the group of shower gels, shower baths, tooth-cleaning agents, mouthwashes, hair shampoos, hair conditioners, conditioning shampoos, hairsprays, hair rinses, hair treatments, hair packings, hair tonics, perming-fixing solutions, hair coloring shampoos, hair colorants, hair-setting compositions, hair arranging compositions, hair styling preparations, blow-dry lotions, mousses, hair gels, hair waxes or combinations thereof.

Particularly preferred cosmetic compositions serve to treat keratin fibers and are therefore hair treatment compositions. In the context of the present invention, hair treatment compositions are, for example, hair shampoos, hair conditioners, conditioning shampoos, hairsprays, hair rinses, hair treatments, hair packings, hair tonics, perming-fixing solutions, hair coloring shampoos, hair colorants, hair-setting compositions, hair arranging compositions, hair styling preparations, blow-dry lotions, mousses, hair gels, hair waxes or combinations thereof. With regard to the fact that men in particular often eschew the use of several different compositions and/or several application steps, preferred compositions are those which men use already. Preferred compositions, therefore, are shampoos, hair conditioners or hair tonics.

The compositions comprise—based on their weight—0.0001 to 20% by weight of at least one selected polyorganosiloxane.

The compositions comprise as first essential ingredient at least one polyorganosiloxane of the formula (I)

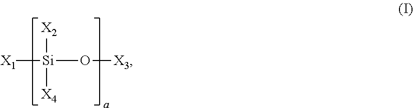

in which
X₁ and X₂ are mutually independently OH, OR¹, R², O-PDMS or O-fSiloxane,
X₃ is hydrogen or a monovalent hydrocarbon radical having 1 to 8 carbon atoms per radical, PDMS or fSiloxane,
X₄ is a radical of the formula

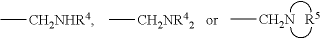

and
a is a number from 1 to 100, preferably a number from 1 to 5,
where
R¹ is an alkyl radical having 1 to 8 carbon atoms,
R² is a monovalent saturated or unsaturated hydrocarbon radical having 1 to 200 carbon atoms per radical, optionally substituted with the elements N, P, S, O, Si and halogen,

### PDMS is

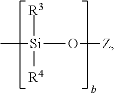

fSiloxane is

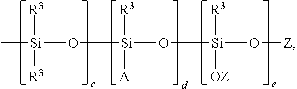

R³ is each mutually independently a monovalent saturated or unsaturated hydrocarbon radical having 1 to 200 carbon atoms per radical, optionally substituted with the elements N, P, S, O, Si and halogen,
A is a radical of the formula R⁶—[NR⁷—R⁸—]_{f}NR⁷₂,
where
R⁶ is a divalent linear or branched hydrocarbon radical having 3 to 18 carbon atoms,
R⁷ is a hydrogen atom, an alkyl radical having 1 to 8 carbon atoms or an acyl radical,
R⁵ is a divalent hydrocarbon radical having 1 to 6 carbon atoms,
b is a number from 1 to 2000, preferably from 1 to 1000,
C is 0 or a number from 1 to 2000, preferably from 50 to 1000,
d is a number from 1 to 1000, preferably from 1 to 10,
e is 0 or a number from 1 to 5,
f is 0, 1, 2, 3 or 4,
Z is hydrogen, an alkyl radical having 1 to 8 carbon atoms or

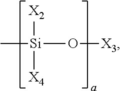

R⁴ is a monovalent hydrocarbon radical having 1 to 18 carbon atoms comprising optionally N and/or O atoms and
R⁵ is a divalent hydrocarbon radical having 3 to 12 carbon atoms comprising optionally N and/or O atoms.

Examples of an alkyl radical R¹ are methyl-, ethyl-, n-propyl-, isopropyl-, 1-n-butyl-, 2-n-butyl-, isobutyl-, tert-butyl-, n-pentyl-, isopentyl-, neopentyl-, tert-pentyl, n-hexyl, n-heptyl, n-octyl, isooctyl or 2,2,4-trimethylpentyl-, where methyl-, ethyl- and butyl- are preferred.

Examples of hydrocarbon radicals R² and R³ are alkyl radicals such as methyl-, ethyl-, n-propyl-, isopropyl-, 1-n-butyl-, 2-n-butyl-, isobutyl-, tert-butyl-, n-pentyl-, isopentyl-, neopentyl-, tert-pentyl, n-hexyl, n-heptyl, n-octyl, isooctyl, 2,2,4-trimethylpentyl-, n-nonyl-, n-decyl-, n-dodecyl-, n-octadecyl-, cyclopentyl-, cyclohexyl-, cycloheptyl-, methylcyclohexyl-, vinyl-, 5-hexenyl-, cyclohexenyl-, 1-propenyl-, allyl-, 3-butenyl-, 4-pentenyl-, phenyl-, naphthyl-, anthryl-, phenanthryl-, o-tolyl-, m-tolyl, p-tolyl-, xylyl-, ethylphenyl-, benzyl-, alpha-phenylethyl- and beta-phenylethyl radicals. Methyl-, ethyl-, octyl- and phenyl radicals are preferred as radical R², particular preference being given to the methyl- and ethyl radical.

Examples of halogenated radicals R² and R³ are the 3,3,3-trifluoro-n-propyl-, 2,2,2,2′,2′,2′-hexafluoroisopropyl-, heptafluoroisopropyl-, o-chlorophenyl-, m-chlorophenyl- and p-chlorophenyl radical.

Examples of R⁴ are the alkyl-, cycloalkyl-, aryl-, alkaryl- and aralkyl radicals listed for the hydrocarbon radicals R² and R³.

Preferred examples of R⁵ are radicals of the formulae —CH₂—CH₂—O—CH₂—CH₂—, —CH₂—CH₂—NH—CH₂—CH₂— or —CH₂—CH₂—NH—CH₂—, where the radical —CH₂—CH₂—O—CH₂—CH₂— is particularly preferred.

Examples of R⁶ are alkylene radicals having 3 to 10 carbon atoms such as propylene, butylene, pentylene, hexylene, heptylene, octylene, nonylene and decylene.

R⁷ can be a hydrogen atom, a methyl-, ethyl-, n-propyl-, isopropyl-, 1-n-butyl-, 2-n-butyl-, isobutyl-, tert-butyl-, n-pentyl-, isopentyl-, neopentyl-, tert-pentyl, n-hexyl, n-heptyl, n-octyl, isooctyl, 2,2,4-trimethylpentyl- or acetyl radical, where preference is given to a hydrogen atom.

Preferred examples for R⁸ are alkylene radicals such as methylene, ethylene, propylene, butylene, pentylene or hexylene.

Z is preferably hydrogen or methyl-, ethyl-, n-propyl-, isopropyl-, 1-n-butyl-, 2-n-butyl-, isobutyl-, tert-butyl-, n-pentyl-, isopentyl-, neopentyl-, tert-pentyl, n-hexyl, n-heptyl, n-octyl, isooctyl or 2,2,4-trimethylpentyl-, where hydrogen, methyl-, ethyl- and butyl- are particularly preferred.

Preferred radicals X₄ corresponding to the above definitions for R⁴ and R⁵ are aminomethyl-, methylaminomethyl-, dimethylaminomethyl-, diethylaminomethyl-, dibutylaminomethyl-, cyclohexylaminomethyl-, morpholinomethyl-, piperidinomethyl-, piperazinomethyl-, ((diethoxymethylsilyl)methyl)cyclohexylaminomethyl-, ((triethoxysilyl)methyl)cyclohexylaminomethyl-, anilinomethyl-, 3-dimethylaminopropylaminomethyl-, bis(3-dimethylaminopropyl)aminomethyl radicals and mixtures thereof. In this case, it is most preferred if the hair treatment composition comprises polyorganosiloxanes of the formula (I), which comprise the morpholinomethyl radical as radical X₄.

In accordance with the definitions of R⁶, R⁷ and R⁸, preferred examples of radical A are:

—(CH₂)₃NH₂
—(CH₂)₃—NH—(CH₂)₂—NH₂
—CH₂CH(CH₃)CH₂—NH—(CH₂)₂—NH₂
—(CH₂)₃—NH(cyclohexyl)
—(CH₂)₃—NHCH₃
—(CH₂)₃—N(CH₃)₂
—(CH₂)₃—NHCH₂CH₃
—(CH₂)₃—N(CH₂CH₃)₂
—(CH₁)₄—NH₂
—CH₂CH(CH₃)CH₂—NH₂
—(CH₂)₃—NH—(CH₂)₂—NHCH₃
—(CH₂)₃—NH—(CH₂)₂—N(CH₃)₂
—(CH₂)₃—NH—(CH₂)₂—NHCH₂CH₃
—(CH₂)₃—NH—(CH₂)₂—N(CH₂CH₃)₂
—(CH₂)₃[—NH—CH₂CH₂]₂—NH₂
—(CH₂)₃—NH(acetyl)
—(CH₂)₃—NH—(CH₂)₂—NH(acetyl) and
—(CH₂)₃—N(acetyl)-(CH₂)₂—NH(acetyl).

To prepare polyorganosiloxanes of the formula (I), preferably commercially available polydimethylsiloxanes having terminal silanol groups and/or polydimethylsiloxanes having terminal alkoxy and silanol groups and/or amine-functionalized siloxanes, which comprise silanol groups or alkoxy and silanol groups, are reacted with a dialkoxy- and/or trialkoxysilane which has a radical of formula

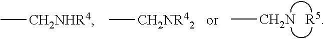

Accordingly in formula (I) “fSiloxane” is a radical which is derived from an amine-functionalized siloxane.

Particular preference is given to using trialkoxysilanes or a mixture of dialkoxy- and trialkoxysilanes, where the use of trialkoxysilanes alone is particularly preferred. When using trialkoxysilanes or a mixture of dialkoxy- and trialkoxysilanes, at least partially crosslinked polyorganopolysiloxanes are obtained independently of the structure of the siloxanes used and the position of the alkoxy and/or silanol groups in the siloxanes. In an especially preferred embodiment, the cosmetic composition comprises crosslinked polyorganosiloxanes. In a most preferred embodiment, the cosmetic composition comprises crosslinked polyorganosiloxanes which are derived from reacting siloxanes and trialkoxysiloxanes.

Preferred examples of dialkoxy- or trialkoxysilanes used comprise:
diethylaminomethylmethyldimethoxysilane,
dibutylaminomethyltriethoxysilane,
dibutylaminomethyltributoxysilane,
cyclohexylaminomethyltrimethoxysilane,
cyclohexylaminomethyltriethoxysilane,
cyclohexylaminomethylmethyldiethoxysilane,
anilinomethyltriethoxysilane,
anilinomethylmethyldiethoxysilane,
morpholinomethyltriethoxysilane,
morpholinomethyltrimethoxysilane,
morpholinomethyltriisopropoxysilane,
3-dimethylaminopropylaminomethyltrimethoxysilane,
morpholinomethyltributoxysilane,
morpholinomethyltrialkoxysilane, wherein the alkoxy radical is a C₁-C₄-alkoxy radical, especially a mixture of methoxy- and ethoxy radicals,
piperazinomethyltriethoxysilane,
piperidinomethyltriethoxysilane and
partial hydrolysates thereof.

A particularly preferred silane is morpholinomethyltriethoxysilane.

A particularly preferred amine-functionalized siloxane that can be used is a copolymer of 3-(2-aminoethylamino)propylmethylsiloxy- and dimethylsiloxy units, which has silanol groups or alkoxy and silanol groups.

In the preparation of the polyorganosiloxanes of the formula (I), identical or different siloxanes and identical or different silanes are used.

A particularly preferred polyorganosiloxane is, for example, known under the INCI name Amodimethicone/Morpholinomethyl Silsesquioxane Copolymer and/or by the CAS number 1293390-78-9. This polyorganosiloxane is commercially available under the name Belsil® ADM 8301 E (ex Wacker). The raw material is a microemulsion and has the following constituents: amodimethicone/morpholinomethyl silsesquioxane copolymer, Trideceth-5, glycerol, phenoxyethanol and water.

The polyorganosiloxanes may be used in varied amounts depending on the application purpose of the cosmetic composition. The cosmetic compositions comprise the polyorganosiloxane in an amount of 0.01% to 10.0% by weight and more preferably from 0.05% to 2.0% by weight, in each case based on the weight of the ready for use composition.

The polyorganosiloxanes are preferably used as aqueous suspensions or aqueous emulsions of polyorganosiloxanes. The dispersions may comprise one or more surfactants as dispersants. The surfactants may be of any type, ionic and/or non-ionic. Alternatively, it is possible to use inorganic solids such as silicas and/or bentonites as dispersants. The average particle size of the polyorganosiloxanes measured by means of light scattering in the dispersions is preferably in the range of 0.001 to 100 μm, more preferably 0.002 to 10 μm. The pH may vary from 1 to 14. The pH is preferably from 3 to 9, particularly preferably from 5 to 8.

The compositions comprise as second essential ingredient—based on its weight—0.0001 to 30% by weight methionylmethionine.

Methionylmethionine is the dipeptide of methionine and has the following structure:

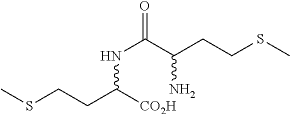

From the dipeptide methionylmethionine exist the four different stereoisomers DD, LL, DL and LD. Of the four stereoisomers only L-methionyl-L-methionine is natural, the three other dipeptides L-methionyl-D-methionine, D-methionyl-L-methionine and D-methionyl-D-methionine all being unnatural. The positive effects described can be achieved with all four stereoisomeric dipeptides. The methionylmethionine may be used as a DD/LL/LD/DL mixture, as a DL/LD mixture or DD/LL mixture. In principle, the use of one stereoisomeric dipeptide alone or in combination with one or two further stereoisomers is also conceivable. If only one stereoisomeric dipeptide is used, preferably L-methionyl-L-methionine or D-methionyl-D-methionine are selected.

The two pairs DD/LL-methionylmethionine and DL/LD-methionylmethionine are diastereomeric relative to one another and have different physical data. For instance, the diastereomeric pair DD/LL has a solubility at room temperature of 21.0 g/L in water, whereas the solubility of the diastereomeric pair DL/LD is 0.4 g/L. Accordingly, it may be preferable to use the methionylmethionine as a DD/LL mixture or as a DD/LL/LD/DL mixture with a high proportion of the DD/LL diastereomeric pair. These mixtures can be incorporated/dissolved simply in an aqueous phase or an aqueous surfactant phase of a cosmetic composition.

The cosmetic compositions comprise methionylmethionine preferably in an amount of 0.01% to 5.0% by weight and particularly preferably from 0.1% to 1.0% by weight, in each case based on the weight of the ready for use cosmetic composition.

It has been shown that the use of the polyorganosiloxanes mentioned above in combination with methionylmethionine imparts excellent properties to the cosmetic compositions. The cosmetic compositions in the form of hair treatment compositions impart more elasticity to the hair treated therewith, which is reflected in higher tensile strengths of the keratin fibers and in a reduction of the elasticity decrease, for example, in the case of damage due to atmospheric effects.

In particular, the particularly preferred morpholinomethyl-containing polyorganosiloxanes in addition in combination with methionylmethionine stabilize the moisture content of the keratin fibers such that the combability is improved and the ageing process is delayed. A further advantage of the cosmetic compositions in the form of hair treatment compositions is that the shapeability and restructurability of keratin fibers treated therewith is improved. Likewise, hydrophobization of the hair surface is effected by the combination of polyorganosiloxanes and methionylmethionine. By means of the hydrophobization, the contact angle of water droplets, which form on the treated hair, is significantly increased, which is a measure of the product performance, particularly the care performance.

Moreover, the cosmetic compositions on application to the skin (for example by a foam bath or shower gel) lead to an improved suppleness of the skin.

The cosmetic compositions may include further active ingredients and auxiliaries. These are described below.

It has been shown that the effect of the cosmetic compositions with the selected polyorganosiloxanes and methionylmethionine can still be increased if certain non-ionic components are also used in the compositions. In addition, these non-ionic components have positive effects on the storage stability of the compositions. Non-ionic components, which are particularly suitable here, are ethoxylates of decanol, undecanol, dodecanol, tridecanol and so on.

Ethoxylated tridecanols have proven to be particularly suitable which are incorporated particularly preferably in the composition. Particularly preferred cosmetic compositions comprise—based on their weight—0.00001 to 5% by weight, preferably 0.0001 to 3.5% by weight, particularly preferably 0.001 to 2% by weight, further preferably 0.01 to 1% by weight, and especially 0.1 to 0.5% by weight, branched ethoxylated tridecanol (INCI name: Trideceth-5) or α-isotridecyl-ω-hydroxypolyglycol ether (INCI name: Trideceth-10) or mixtures thereof.

Preferred cosmetic compositions are characterized in that they comprise—based on their weight—0.5 to 70% by weight, preferably 1 to 60% by weight, and especially 5 to 25% by weight anionic and/or non-ionic and/or cationic and/or amphoteric surfactant(s).

Anionic surfactants suitable for the cosmetic compositions are all anionic surface-active substances suitable for use on the human body. These are characterized by a water-solubilizing anionic group such as a carboxylate, sulphate, sulphonate or phosphate group and a lipophilic alkyl group having about 8 to 30 carbon atoms. In addition, glycol or polyglycol ether groups, ester, ether and amide groups and also hydroxyl groups may be present in the molecule.

Amphoteric surfactants, which are also referred to as zwitterionic surfactants, are those surface-active compounds mentioned which bear at least one quaternary ammonium group and at least one —COO⁽⁻⁾ or —SO₃⁽⁻⁾ group in the molecule. Particularly suitable amphoteric/zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxylmethyl-3-hydroxyethylimidazolines each having 8 to 18 carbon atoms in the alkyl or acyl group, as well as cocoacylaminoethylhydroxyethyl carboxymethyl glycinate. A preferred amphoteric/zwitterionic surfactant is the fatty acid amide derivative known under the INCI name Cocamidopropyl Betaine.

Amphoteric surfactants are also understood to mean those surface-active compounds which, apart from a C₈-C₂₄-alkyl or -acyl group, comprise at least one free amino group and at least one —COOH or —SO₃H group and are capable of forming internal salts. Examples of suitable amphoteric/zwitterionic surfactants are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each having about 8 to 24 carbon atoms in the alkyl group. Particularly preferred amphoteric/zwitterionic surfactants are N-cocoalkylaminopropionate, cocoacylaminoethylaminopropionate and C₁₂-C₁₈-acylsarcosine.

Nonionic surfactants comprise as hydrophilic group, for example, a polyol group, a polyalkylene glycol ether group or a combination of polyol and polyglycol ether groups.

Especially preferred are cosmetic compositions which additionally comprise fatty alcohol(s) and/or fatty alcohol alkoxylate(s), preferably C₁₂₋₂₂-fatty alcohol(s) and/or C₁₂₋₂₂-fatty alcohol ethoxylate(s) having 10 to 30 EO units, particularly preferably C₁₆₋₁₈-fatty alcohol(s) and/or C₁₆₋₁₈-fatty alcohol ethoxylate(s) having 12 to 20 EO units, preferably in amounts of 5 to 20% by weight, preferably 7.5 to 17.5% by weight and especially 10 to 15% by weight, in each case based on the weight of the cosmetic composition.

In summary, cosmetic compositions, particularly hair treatment compositions, are preferred which comprise—based on their weight—0.1 to 20% by weight, preferably 0.25 to 17.5% by weight and especially 5 to 15% by weight anionic surfactant(s), particularly preferably fatty alcohol ether sulphates of the formula

H₃C—(CH₂)ₙ—(OCH₂CH₂)ₖ—OSO₃⁻ M⁺

in which n is a value from 5 to 21, preferably from 7 to 19, particularly preferably from 9 to 17 and especially from 11 to 13 and k is a value of 1, 2, 3, 4, 5, 6, 7, 8, 9 or 10, preferably 1, 2 or 3 and especially 2, and M is a cation from the group of Na⁺, K⁺ NH₄⁺, ½Mg²⁺, ½Zn²⁺, preferably Na⁺.

As further optional constituent, the cosmetic compositions may comprise 0.01 to 10% by weight of at least one polymer from the group of cationic and/or amphoteric polymers.

Cationic or amphoteric polymers are understood to mean polymers which have a group in the main and/or side chain, which may be “temporarily” or “permanently” cationic. Polymers referred to as “permanently cationic” are those having a cationic group independently of the pH of the composition. These are generally polymers comprising a quaternary nitrogen atom, in the form of an ammonium group for example. Preferred cationic groups are quaternary ammonium groups. Those polymers in particular, in which the quaternary ammonium groups are linked via a C1-4-hydrocarbon group to a main polymer chain constructed from acrylic acid, methacrylic acid or derivatives thereof have proven to be particularly suitable.

In addition to cationic polymers or in place thereof, the hair treatment compositions may also comprise amphoteric polymers. In addition to the at least one cationically charged group, these have at least one negatively charged group in the molecule and are also referred to as zwitterionic polymers.

The polymer(s) is/are preferably used in an amount within a narrow range. For instance, cosmetic compositions are preferred which comprise—based on their weight—0.05 to 7.5% by weight, preferably 0.1 to 5% by weight, particularly preferably 0.2 to 3.5% by weight and especially 0.25 to 2.5% by weight amphoteric polymer(s).

Independently of whether amphoteric polymers are present in the compositions or not, preferred cosmetic compositions are further characterized in that they comprise—based on their weight—0.05 to 7.5% by weight, preferably 0.1 to 5% by weight, particularly preferably 0.2 to 3.5% by weight, and especially 0.25 to 2.5% by weight cationic polymer(s).

Further suitable ingredients comprise non-ionic polymers, anionic polymers, lipids, waxes, protein hydrolysates, amino acids, oligopeptides, vitamins, provitamins, vitamin precursors, betaines, bioquinones, purine (derivatives), taurine (derivatives), care substances, vegetable extracts, ester oils, UV light protection filters, structurants, thickeners, electrolytes, pH modifiers, swelling agents, dyes, antidandruff active ingredients, complexing agents, opacifiers, pearlizing agents, pigments, stabilizers, blowing agents, antioxidants, perfume oils, further silicones and/or preservatives.

The cosmetic compositions have advantageous properties and likewise impart advantageous properties to the body surfaces and/or hair treated therewith. In the hair and scalp treatment in particular, advantages were observed which surpass the advantages of the polyorganosiloxanes or methionylmethionine alone. For instance, the hair treatment compositions increase the elasticity of the hair treated therewith and result in an internal structural strengthening of the hair fibers which is reflected, for example, in relatively high melting temperatures in the differential thermal analysis. This also results in hydrophobization of the hair surface which is revealed in an increase of the contact angle of water droplets on the hair surface. It is also revealed by an improvement of the wet and dry combability. On the skin and particularly the scalp, the cosmetic compositions cause an increase in the elasticity and, surprisingly, sebum-regulating effects. The optical impression of “greasy” skin or hair is therefore avoided or diminished.

The invention further relates to the use of methionylmethionine in a cosmetic composition, preferably in a hair treatment composition, comprising polyorganosiloxanes of the formula (I)

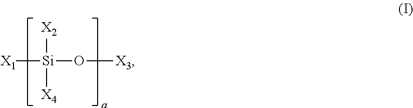

in which
X₁ and X₂ are mutually independently OH, OR¹, R², O-PDMS or O-fSiloxane,
X₃ is hydrogen or a monovalent hydrocarbon radical having 1 to 8 carbon atoms per radical, PDMS or fSiloxane,
X₄ is a radical of the formula

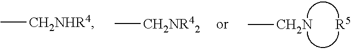

and
a is a number from 1 to 100,
where
R¹ is an alkyl radical having 1 to 8 carbon atoms,
R² is a monovalent saturated or unsaturated hydrocarbon radical having 1 to 200 carbon atoms per radical, optionally substituted with the elements N, P, S, O, Si and halogen,

### PDMS is

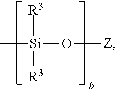

fSiloxane is

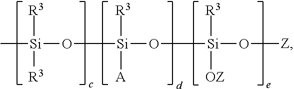

R³ is each mutually independently a monovalent saturated or unsaturated hydrocarbon radical having 1 to 200 carbon atoms per radical, optionally substituted with the elements N, P, S, O, Si and halogen,
A is a radical of the formula R⁶—[NR⁷—R⁸—]_{f}NR⁷₂,
where
R⁶ is a divalent linear or branched hydrocarbon radical having 3 to 18 carbon atoms,
R⁷ is a hydrogen atom, an alkyl radical having 1 to 8 carbon atoms or an acyl radical.
R⁸ is a divalent hydrocarbon radical having 1 to 6 carbon atoms,
b is a number from 1 to 2000,
c is 0 or a number from 1 to 2000.
d is a number from 1 to 1000,
e is 0 or a number from 1 to 5,
f is 0, 1, 2, 3 or 4,
Z is hydrogen, an alkyl radical having 1 to 8 carbon atoms or

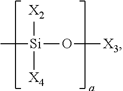

R⁴ is a monovalent hydrocarbon radical having 1 to 18 carbon atoms comprising optionally N and/or O atoms and
R⁵ is a divalent hydrocarbon radical having 3 to 12 carbon atoms comprising optionally N and/or O atoms,
for improving at least one of the properties
tensile strength of keratin fibers, particularly of human hair;
combability of keratin fibers, particularly of human hair;
hydrophobizing the surface of keratin fibers, particularly of human hair;
strengthening the internal structure of keratin fibers, particularly of human hair;
sebum regulation of the skin, particularly the scalp.

For this purpose, the cosmetic composition is applied to the keratin fibers and/or the skin, preferably the scalp, left there for 5 seconds to 10 minutes and then rinsed off, or the cosmetic composition is applied to the keratin fibers and/or the skin, preferably the scalp, and remains there.

With regard to further preferred embodiments of the use, the statements made apply, mutatis mutandis, to the cosmetic compositions.

The following examples are intended to illustrate the subject matter of the present invention but without restricting it.

### EXAMPLES

All quantitative data, unless otherwise stated, are parts by weight of active substance. The following formulations were provided using known preparation processes.

Hair conditioner:

| | % by |
|---|---|
| | weight |
| Cetearyl Alcohol | 5.0 |
| Quaternium-87 | 0.75 |
| Glycol Distearate | 1.0 |
| Amodimethicone/Morpholinomethyl Silsesquioxane | 0.3 |
| Copolymer, Trideceth-5, Glycerol | |
| Shea Butter (INCI: Butyrospermum Parkii (Shea) | 1.5 |
| Butter) | |
| L-methionyl-L-methionine | 1.0 |
| Lactic acid | 0.1 |
| Behenoyl PG—Trimoniumchloride | 1.5 |
| Behentrimonium Chloride | 0.5 |
| Polyquaternium-37 | 0.25 |
| Glycerol | 0.1 |
| Dimethicone | 1.0 |
| Water, preservative, accompanying substances and | to 100 |
| optionally perfume oils | |

Care spray:

| | % by |
|---|---|
| | weight |
| Stearamidopropyldimethylamine | 0.3 |
| Glycerol | 2.0 |
| Polyquaternium-10 | 0.2 |
| Amodimethicone/Morpholinomethyl Silsesquioxane | 0.02 |
| Copolymer | |
| Panthenol | 0.2 |
| DL-Methionyl-DL-methionine | 0.8 |
| Ethanol | 15 |
| Nicotinamide | 0.1 |
| Dicaprylyl Carbonate | 0.1 |
| Cetrimonium Chloride | 0.2 |
| Water, preservative, accompanying substances and | to 100 |
| optionally perfume oils | |

Hair shampoo:

| | % by |
|---|---|
| | weight |
| Sodium Laureth Sulfate | 9.0 |
| Ammonium Lauryl Sulfate | 5.0 |
| Disodium Cocoamphodiacetate | 2.0 |
| Cocoamidopropyl Betaine | 6.0 |
| D-Methionyl-D-methionine | 0.5 |
| Panthenol | 0.2 |
| Amodimethicone/Morpholinomethyl | 0.06 |
| Silsesquioxane Copolymer, Trideceth-5, | |
| Glycerol | |
| Citric acid | 0.4 |
| Guar Hydroxypropyltrimonium | 0.4 |
| Chloride | |
| Sodium chloride | 1.3 |
| Water, preservative, accompanying | to 100 |
| substances and optionally perfume oils | |

The following commercial products were used:

| Commercial product | INCI | Supplier/manufacturer |
|---|---|---|
| Lanette ® O | Cetearyl Alcohol | BASF SE |
| Varisoft W575 PG | Quaternium-87 | Evonik |
| Cutina ® GMS-V or Cutina ® | Glycol Distearate | BASF SE |
| AGS | | |
| Polymer JR400 | Polyquaternium-10 | Dow |
| Dehyquart ® A CA | Cetrimonium Chloride | BASF SE |
| Synthalen ® CR | Polyquaternium-37 | 3 V Sigma |
| Quartamin ® BTC 131 | Behenoyl PG—Trimoniumchloride | Kao Chemicals |
| Genamin ® KDMP | Behentrimonium Chloride | Clariant |
| Tego Amid S 18 | Stearamidopropyldimethylamine | Evonik |
| Ucare ® Polymer JR 400 | Polyquaternium-10 | Dow |
| Cetiol CC | Dicaprylyl Carbonate | BASF SE |
| Texapon N70 NA | Sodium Laureth Sulfate | BASF SE |
| Empicol AL 70 | Ammonium Lauryl Sulfate | Huntsman |
| Rewoteric AM 2 | Disodium Cocoamphodiacetate | Evonik |
| Tego Betain F50 | Cocoamidopropyl Betaine | Evonik |
| N-Hance 3196 | Guar Hydroxypropyltrimonium | Ashland |
| | Chloride | |
| Belsil ® ADM 8301 E | Amodimethicone/Morpholinomethyl | Wacker Silicones |
| | Silsesquioxane Copolymer, Trideceth- | |
| | 5, Glycerol | |

1. A cosmetic composition comprising—in each case based on its weight—
a) 0.0001% to 20% by weight polyorganosiloxanes of the formula (I) 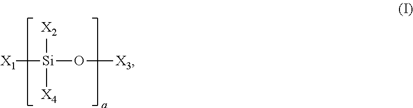
in which
X₁ and X₂ are mutually independently OH, OR¹, R², O-PDMS or O-fSiloxane,
X₃ is hydrogen or a monovalent hydrocarbon radical having 1 to 8 carbon atoms per radical, PDMS or fSiloxane,
X₄ is a radical of the formula 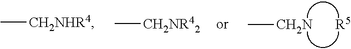
and
a is a number from 1 to 100,
where
R¹ is an alkyl radical having 1 to 8 carbon atoms,
R² is a monovalent saturated or unsaturated hydrocarbon radical having 1 to 200 carbon atoms per radical, optionally substituted with the elements N, P, S, O, Si and halogen,
PDMS is 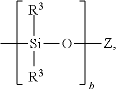
fSiloxane is 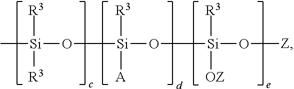
R³ is each mutually independently a monovalent saturated or unsaturated hydrocarbon radical having 1 to 200 carbon atoms per radical, optionally substituted with the elements N, P, S, O, Si and halogen,
A is a radical of the formula R⁶—[NR⁷—R⁸—]_{f}NR⁷₂,
where
R⁶ is a divalent linear or branched hydrocarbon radical having 3 to 18 carbon atoms,
R⁷ is a hydrogen atom, an alkyl radical having 1 to 8 carbon atoms or an acyl radical,
R⁸ is a divalent hydrocarbon radical having 1 to 6 carbon atoms,
b is a number from 1 to 2000,
c is 0 or a number from 1 to 2000,
d is a number from 1 to 1000,
e is 0 or a number from 1 to 5,
f is 0, 1, 2, 3 or 4,
Z is hydrogen, an alkyl radical having 1 to 8 carbon atoms or 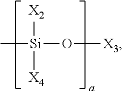
R⁴ is a monovalent hydrocarbon radical having 1 to 18 carbon atoms comprising optionally N and/or O atoms and
R⁵ is a divalent hydrocarbon radical having 3 to 12 carbon atoms comprising optionally N and/or O atoms, and
b) 0.0001 to 30% by weight methionylmethionine.

2. The cosmetic composition according to claim 1, characterized in that fSiloxan is an amine-functionalized siloxane, preferably a copolymer of 3-(2-aminoethylamino)propylmethylsiloxy- and dimethylsiloxy units.

3. The cosmetic composition according to claim 1, wherein X₄ is selected from the group consisting of aminomethyl-, methylaminomethyl-, dimethylaminomethyl-, diethylaminomethyl-, dibutylaminomethyl-, cyclohexylaminomethyl-, morpholinomethyl-, piperidinomethyl-, piperazinomethyl-, ((diethoxymethylsilyl)methyl)cyclohexylaminomethyl-, ((triethoxysilyl)methyl)cyclohexylaminomethyl-, anilinomethyl-, 3-dimethylaminopropylaminomethyl-, bis(3-dimethylaminopropyl)aminomethyl- and mixtures thereof.

4. The cosmetic composition according to claim 1, wherein the composition comprises polyorganosiloxanes, which comprise a morpholinomethyl radical as radical X₄.

5. The cosmetic composition according to claim 1, wherein the composition—based on its weight—further comprises branched ethoxylated tridecanol (INCI name: Trideceth-5) or α-isotridecyl-ω-hydroxypolyglycol ether (INCI name: Trideceth-10) or mixtures thereof.

6. The cosmetic composition according to claim 1, wherein the methionylmethionine is a mixture of L-methionyl-L-methionine, L-methionyl-D-methionine, D-methionyl-L-methionine and D-methionyl-D-methionine.

7. Hair treatment composition according to claim 1, wherein the methionylmethionine is L-methionyl-L-methionine and/or D-methionyl-D-methionine.

8. A cosmetic composition comprising—in each case based on its weight—
a) 0.0001 to 20% by weight of a polyorganosiloxane, which is known under the INCI name Amodimethicone/Morpholinomethyl Silsesquioxane Copolymer, and
b) 0.0001 to 30% by weight methionylmethionine.

9. A cosmetic composition comprising—in each case based on its weight—
a) 0.0001 to 20% by weight of a polyorganosiloxane, which is obtainable by reacting a siloxane, comprising a silanol group or alkoxy and silanol groups, with a trialkoxysilane or a mixture of dialkoxy- and trialkoxysilanes, wherein the dialkoxy- and trialkoxysilanes have a morpholinomethyl radical, and
b) 0.0001 to 30% by weight methionylmethionine.

10. A cosmetic composition of methionylmethionine comprising polyorganosiloxanes of the formula (I) 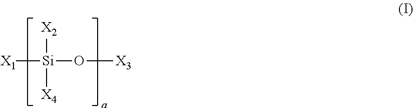
in which
X₁ and X₂ are mutually independently OH, OR¹, R², O-PDMS or O-fSiloxane,
X₃ is hydrogen or a monovalent hydrocarbon radical having 1 to 8 carbon atoms per radical, PDMS or fSiloxane,
X₄ is a radical of the formula 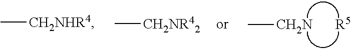
and
a is a number from 1 to 100,
where
R¹ is an alkyl radical having 1 to 8 carbon atoms,
R² is a monovalent saturated or unsaturated hydrocarbon radical having 1 to 200 carbon atoms per radical, optionally substituted with the elements N, P, S, O, Si and halogen,
PDMS is 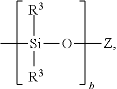
fSiloxane is 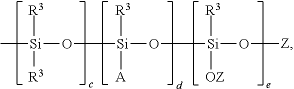
R³ is each mutually independently a monovalent saturated or unsaturated hydrocarbon radical having 1 to 200 carbon atoms per radical, optionally substituted with the elements N, P, S, O, Si and halogen,
A is a radical of the formula R⁶—[NR⁷—R⁸—]_{f}NR⁷₂,
where
R⁶ is a divalent linear or branched hydrocarbon radical having 3 to 18 carbon atoms,
R⁷ is a hydrogen atom, an alkyl radical having 1 to 8 carbon atoms or an acyl radical,
R⁸ is a divalent hydrocarbon radical having 1 to 6 carbon atoms,
b is a number from 1 to 2000,
c is 0 or a number from 1 to 2000,
d is a number from 1 to 1000,
e is 0 or a number from 1 to 5,
f is 0, 1, 2, 3 or 4,
Z is hydrogen, an alkyl radical having 1 to 8 carbon atoms or 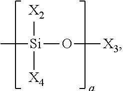
R⁴ is a monovalent hydrocarbon radical having 1 to 18 carbon atoms comprising optionally N and/or O atoms and
R⁵ is a divalent hydrocarbon radical having 3 to 12 carbon atoms comprising optionally N and/or O atoms,
for improving at least one of the properties
tensile strength of keratin fibres, particularly of human hair;
combability of keratin fibres, particularly of human hair;
hydrophobizing the surface of keratin fibres, particularly of human hair;
strengthening the internal structure of keratin fibres, particularly of human hair;
sebum regulation of the skin, particularly the scalp.

11. A hair treatment composition comprising the cosmetic composition of claim 10.

12. The cosmetic composition according to claim 2, wherein X₄ is selected from the group consisting of aminomethyl-, methylaminomethyl-, dimethylaminomethyl-, diethylaminomethyl-, dibutylaminomethyl-, cyclohexylaminomethyl-, morpholinomethyl-, piperidinomethyl-, piperazinomethyl-, ((diethoxymethylsilyl)methyl)cyclohexylaminomethyl-, ((triethoxysilyl)methyl)cyclohexylaminomethyl-, anilinomethyl-, 3-dimethylaminopropylaminomethyl-, bis(3-dimethylaminopropyl)aminomethyl- and mixtures thereof.

13. The cosmetic composition according to claim 2, wherein the composition comprises polyorganosiloxanes, which comprise a morpholinomethyl radical as radical X₄.

14. The cosmetic composition according to claim 2, wherein the composition—based on its weight—further comprises branched ethoxylated tridecanol (INCI name: Trideceth-5) or α-isotridecyl-ω-hydroxypolyglycol ether (INCI name: Trideceth-10) or mixtures thereof.

15. The cosmetic composition according to claim 2, wherein the methionylmethionine is a mixture of L-methionyl-L-methionine, L-methionyl-D-methionine, D-methionyl-L-methionine and D-methionyl-D-methionine.

16. Hair treatment composition according to claim 2, wherein the methionylmethionine is L-methionyl-L-methionine and/or D-methionyl-D-methionine.

17. The cosmetic composition according to claim 3, wherein the composition comprises polyorganosiloxanes, which comprise a morpholinomethyl radical as radical X₄.

18. The cosmetic composition according to claim 3, wherein the composition—based on its weight—further comprises branched ethoxylated tridecanol (INCI name: Trideceth-5) or α-isotridecyl-ω-hydroxypolyglycol ether (INCI name: Trideceth-10) or mixtures thereof.

19. The cosmetic composition according to claim 3, wherein the methionylmethionine is a mixture of L-methionyl-L-methionine, L-methionyl-D-methionine, D-methionyl-L-methionine and D-methionyl-D-methionine.

20. Hair treatment composition according to claim 3, wherein the methionylmethionine is L-methionyl-L-methionine and/or D-methionyl-D-methionine.
